(19) United States
(12) Patent Application Publication
(10) Pub. No.: US 2025/0120601 A1
(43) Pub. Date: Apr. 17, 2025

(54) BLOOD-PRESSURE GAUGE AND METHOD FOR MEASURING BLOOD PRESSURE

(71) Applicant: OMRON HEALTHCARE Co., Ltd., Kyoto (JP)

(72) Inventors: Shingo YAMASHITA, Kyoto (JP); Yuka SUZUKI, Kyoto (JP); Yukiya SAWANOI, Kyoto (JP)

(21) Appl. No.: 18/990,425

(22) Filed: Dec. 20, 2024

Related U.S. Application Data
(63) Continuation of application No. PCT/JP2023/018714, filed on May 19, 2023.

(30) Foreign Application Priority Data
Sep. 7, 2022 (JP) 2022-142431

(51) Int. Cl. A61B 5/022; A61B 5/00; A61B 5/021; A61B 5/024; G16H 40/63

(52) U.S. Cl. CPC A61B 5/02225; A61B 5/02108; A61B 5/02444; A61B 5/7264; A61B 5/7282; G16H 40/63; A61B 2562/0247

(57) ABSTRACT

A blood-pressure gauge includes a blood pressure measurement unit that automatically performs blood pressure measurement according to a predetermined schedule in one of a plurality of blood pressure measurement modes. The plurality of blood pressure measurement modes include a normal measurement mode and a plurality of measurement modes having higher reliability than the normal measurement mode. The blood-pressure gauge further includes a determination unit that determines whether any of a plurality of events has occurred in a user during blood pressure measurement, and a mode setting unit that sets an active measurement mode from among the plurality of blood pressure measurement modes based on a determination result. The mode setting unit sets the normal measurement mode as the active measurement mode when no event has occurred, and sets one of the plurality of measurement modes as the active measurement mode when one of the events has occurred.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/018714, filed May 19, 2023, which application claims priority to Japanese Patent Application No. 2022-142431, filed Sep. 7, 2022, which applications are incorporated herein by reference in their entireties.

### TECHNICAL FIELD

The present disclosure relates to a blood-pressure gauge and a method for measuring a blood pressure.

### BACKGROUND ART

A blood-pressure gauge having a night (sleep) blood-pressure measurement mode is known in the related art. For example, Patent Document 1 (JP 2021-69444 A) discloses a technique of appropriately setting a re-measurement time in accordance with an event that has occurred in a subject when there is a possibility that a current blood pressure value measured in the night blood-pressure measurement mode has a measurement error.

### CITATION LIST

### Patent Literature

Patent Document 1: JP 2021-69444 A

### SUMMARY OF INVENTION

### Technical Problem

When blood pressure is measured over a long period of time (for example, overnight during sleep or the like), various events that may affect blood pressure measurement, such as occurrence of irregular pulse waves and body motions, may occur to a subject. When such an event occurs, there is a possibility that a blood pressure value cannot be appropriately measured during blood pressure measurement. In addition, when an irregular pulse wave is detected during blood pressure measurement, it is desirable to determine whether the irregular pulse wave is an arrhythmia such as atrial fibrillation. Thus, there is a demand for a technique for improving the reliability of measurement results (for example, blood pressure values, the occurrence of an arrhythmia, etc.) obtained from blood pressure measurement that automatically starts while a subject is sleeping or the like.

The present disclosure aims in some respects to provide a blood-pressure gauge and a method for measuring a blood pressure that can improve the reliability in measurement results when blood pressure measurement automatically starts.

### Solution to Problem

In an example of the present disclosure, a blood-pressure gauge that measures a blood pressure by compressing a measurement site of a user with a cuff is provided. The blood-pressure gauge includes a blood pressure measurement unit that automatically performs blood pressure measurement in one of a plurality of blood pressure measurement modes according to a predetermined schedule. The plurality of blood pressure measurement modes include a normal measurement mode and a plurality of measurement modes having higher reliability in measurement results than the normal measurement mode. The blood-pressure gauge further includes a determination unit that determines whether any of a plurality of predetermined events has occurred in a user while the blood pressure measurement unit was measuring a blood pressure, and a mode setting unit that sets an active measurement mode to be performed by the blood pressure measurement unit from among the plurality of blood pressure measurement modes based on a determination result of the determination unit. The mode setting unit sets the normal measurement mode as the active measurement mode when none of the plurality of events has occurred, and sets one of the plurality of measurement modes as the active measurement mode when one of the plurality of events has occurred.

According to the above configuration, it is possible to improve the reliability in measurement results when blood pressure measurement automatically starts.

In another example of the present disclosure, the plurality of measurement modes include a first measurement mode in which the blood pressure measurement is consecutively performed a plurality of times or more. The determination unit determines whether an arrhythmia has occurred in the user based on a pulse wave signal obtained when the blood pressure measurement unit performs blood pressure measurement. The mode setting unit sets the normal measurement mode or the first measurement mode as the active measurement mode based on whether an arrhythmia has occurred.

According to the above configuration, the first measurement mode that has high reliability is set according to whether an arrhythmia has occurred.

In another example of the present disclosure, the mode setting unit sets the normal measurement mode as the active measurement mode when no arrhythmia has occurred, and sets the first measurement mode as the active measurement mode when an arrhythmia has occurred.

According to the above configuration, since the blood pressure measurement in the first measurement mode is performed on the user to whom an arrhythmia has occurred, it is possible to obtain a highly reliable measurement result.

In another example of the present disclosure, the mode setting unit sets the normal measurement mode as the active measurement mode if no arrhythmia has occurred, and sets the first measurement mode as the active measurement mode when the number of times an arrhythmia has occurred in the most recent specified number of times of blood pressure measurement is equal to or greater than a reference number of times if an arrhythmia has occurred.

According to the above configuration, since the blood pressure measurement in the first measurement mode is performed on the user prone to arrhythmias, it is possible to obtain a highly reliable measurement result.

In another example of the present disclosure, the mode setting unit sets the normal measurement mode as the active measurement mode when no arrhythmia has occurred, and sets the first measurement mode as the active measurement mode when arrhythmias have consecutively occurred a predetermined number of times.

According to the above configuration, since the blood pressure measurement in the first measurement mode is performed on the user prone to arrhythmias, it is possible to obtain a highly reliable measurement result.

In another example of the present disclosure, when the first measurement mode is set, the blood pressure measurement unit determines whether a predetermined period has elapsed from the previous blood pressure measurement, performs blood pressure measurement in the first measurement mode when the predetermined period has not elapsed, and performs blood pressure measurement in the normal measurement mode when the predetermined period has elapsed.

According to the above configuration, when the predetermined period has elapsed, it is possible to automatically switch the active measurement mode from the first measurement mode to the normal measurement mode.

In another example of the present disclosure, the plurality of measurement modes include a second measurement mode. In the normal measurement mode, the blood pressure measurement unit measures a blood pressure of a user based on a pulse wave signal in a first pressurization process of increasing a cuff pressure indicating a pressure inside the cuff worn around a measurement site of the user. The determination unit determines whether an irregular pulse wave has occurred in the user based on the pulse wave signal in the first pressurization process. The mode setting unit sets the normal measurement mode as the active measurement mode when no irregular pulse wave has occurred, and sets the second measurement mode when an irregular pulse wave has occurred.

According to the above configuration, since the blood pressure measurement in the second measurement mode is performed on the user to whom an irregular pulse wave has occurred, it is possible to obtain a highly reliable measurement result.

In another example of the present disclosure, in the second measurement mode, the blood pressure measurement unit measures a blood pressure of the user based on the pulse wave signal in a first pressurization process, and continues the first pressurization process until a first pulse rate of the user acquired based on a pulse wave signal in the first pressurization process reaches a threshold or higher. The determination unit determines whether an arrhythmia has occurred in the user based on the pulse wave signal in the first pressurization process.

According to the above configuration, since the blood pressure measurement in the second measurement mode is performed, a larger amount of pulse rates is acquired, and thus arrhythmias can be determined with high reliability.

In another example of the present disclosure, the plurality of measurement modes include a third measurement mode. In the normal measurement mode, the blood pressure measurement unit measures a blood pressure of a user based on a pulse wave signal in a depressurization process of reducing the cuff pressure after the second pressurization process of increasing the cuff pressure indicating a pressure inside the cuff worn around a measurement site of the user to a pressure higher than an estimated systolic blood pressure. The determination unit determines whether an irregular pulse wave has occurred in the user based on the pulse wave signal in the depressurization process. The mode setting unit sets the normal measurement mode as the active measurement mode when no irregular pulse wave has occurred, and sets the third measurement mode when an irregular pulse wave has occurred.

According to the above configuration, since the blood pressure measurement in the third measurement mode is performed on the user to whom an irregular pulse wave has occurred, it is possible to obtain a highly reliable measurement result.

In another example of the present disclosure, in the third measurement mode, the blood pressure measurement unit measures a blood pressure of the user based on the pulse wave signal in the depressurization process after the second pressurization process, and continues the second pressurization process until a second pulse rate of the user acquired based on a pulse wave signal in the second pressurization process reaches a threshold or higher. The determination unit determines whether an arrhythmia has occurred in the user based on the pulse wave signal in the depressurization process.

According to the above configuration, since the blood pressure measurement in the third measurement mode is performed, a larger amount of pulse rates is acquired, and thus arrhythmias can be determined with high reliability.

In another example of the present disclosure, the plurality of measurement modes include a fourth measurement mode in which a blood pressure is re-measured when a user makes a body motion. The determination unit determines whether a body motion has been made based on a pulse wave signal obtained when the blood pressure measurement unit performs blood pressure measurement. The mode setting unit sets the normal measurement mode or the fourth measurement mode as the active measurement mode based on whether a body motion has been made.

According to the above configuration, the fourth measurement mode that has high reliability is set according to whether a body motion has been made.

In another example of the present disclosure, the mode setting unit sets the normal measurement mode as the active measurement mode when no body motion has been made in the current blood pressure measurement, and sets the fourth measurement mode as the active measurement mode when a body motion has been made in the current blood pressure measurement and the cumulative number of times of blood pressure measurement is less than a first number of times.

According to the above configuration, since the blood pressure measurement in the fourth measurement mode is performed on the user who made a body motion, it is possible to obtain a highly reliable measurement result.

In another example of the present disclosure, a method for measuring a blood pressure for a blood-pressure gauge that measures a blood pressure by compressing a measurement site of a user with a cuff is provided. The method for measuring a blood pressure includes a step of automatically performing blood pressure measurement in one of a plurality of blood pressure measurement modes according to a predetermined schedule. The plurality of blood pressure measurement modes include a normal measurement mode and a plurality of measurement modes having higher reliability in measurement results than the normal measurement mode. The method for measuring a blood pressure further includes a step of determining whether any of a plurality of predetermined events has occurred in a user while a blood pressure was being measured, and a step of setting an active measurement mode to be performed in the performing step from among the plurality of blood pressure measurement modes based on a determination result of the determining step. The setting step includes setting a normal measurement mode as an active measurement mode when none of the plurality of events has occurred, and setting one of the plurality of measurement modes as the active measurement mode when one of the plurality of events has occurred.

According to the above configuration, it is possible to improve the reliability in measurement results when blood pressure measurement automatically starts.

### Advantageous Effects of Invention

According to the present disclosure, it is possible to improve the reliability in measurement results when blood pressure measurement automatically starts.

### BRIEF DESCRIPTION OF DRAWINGS

FIG. 1 is a diagram for describing an application example of a blood-pressure gauge.

FIG. 2 is a diagram illustrating an appearance of the blood-pressure gauge.

FIG. 3 is a block diagram illustrating a hardware configuration of the blood-pressure gauge.

FIG. 4 is a block diagram illustrating a functional configuration of the blood-pressure gauge.

FIG. 5 is a flowchart showing an example of a setting process procedure for a measurement mode according to the occurrence of an arrhythmia.

FIG. 6 is a flowchart showing an example of a blood pressure measurement process in a normal mode of the blood-pressure gauge.

FIG. 7 is a flowchart showing another example of the blood pressure measurement process in the normal mode of the blood-pressure gauge.

FIG. 8 is a flowchart showing an example of a blood pressure measurement process in a first measurement mode of the blood-pressure gauge.

FIG. 9 is a flowchart showing another example of the blood pressure measurement process in the first measurement mode of the blood-pressure gauge.

FIG. 10 is a flowchart showing another example of the setting process procedure for a measurement mode according to the occurrence of an arrhythmia.

FIG. 11 is a flowchart showing another example of the setting process procedure for a measurement mode according to the occurrence of an arrhythmia.

FIG. 12 is a flowchart showing an example of a setting process procedure for a measurement mode according to the occurrence of irregular pulse waves.

FIG. 13 is a flowchart showing an example of a blood pressure measurement process in a second measurement mode of the blood-pressure gauge.

FIG. 14 is a flowchart showing another example of the setting process procedure for a measurement mode according to the occurrence of irregular pulse waves.

FIG. 15 is a flowchart showing another example of the setting process procedure for a measurement mode according to the occurrence of irregular pulse waves.

FIG. 16 is a flowchart showing an example of a blood pressure measurement process in a third measurement mode of the blood-pressure gauge.

FIG. 17 is a flowchart showing an example of a setting process procedure for a measurement mode according to the occurrence of body motions.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### APPLICATION EXAMPLE

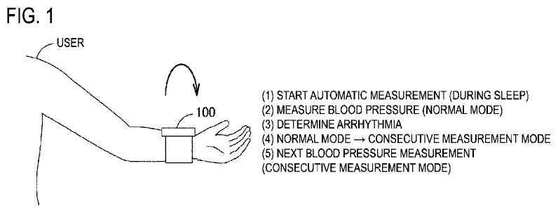

An application example of the present invention will be described with reference to FIG. 1. FIG. 1 is a diagram for describing an application example of a blood-pressure gauge 100.

Referring to FIG. 1, the blood-pressure gauge 100 is a wrist-type blood-pressure gauge that measures a blood pressure by compressing a measurement site of a user (that is, a subject) with a cuff. To measure a blood pressure, the blood-pressure gauge 100 is worn with the cuff wound around the wrist of the user. The blood-pressure gauge 100 includes a main body and a cuff (arm band) as main components. Note that the blood-pressure gauge 100 may be an upper arm-type blood-pressure gauge in which a main body and a cuff (arm band) are integrated. Hereinafter, the processing contents will be described with reference to FIG. 1.

In FIG. 1, it is assumed that the blood-pressure gauge 100 automatically measures a blood pressure of a user while the user is sleeping. The blood-pressure gauge 100 automatically starts measuring a blood pressure according to a predetermined schedule (corresponding to (1) in FIG. 1).

The blood-pressure gauge 100 performs blood pressure measurement of the user by the oscillometric method in a normal measurement mode (hereinafter also simply referred to as a “normal mode”) (corresponding to (2) in FIG. 1). The blood-pressure gauge 100 determines the occurrence of an arrhythmia such as atrial fibrillation, which is one of events that may affect blood pressure measurement, based on a pulse wave signal obtained during blood pressure measurement. Here, it is assumed that an arrhythmia has occurred (corresponding to (3) in FIG. 1).

Subsequently, when an arrhythmia has occurred, the blood-pressure gauge 100 switches the measurement mode that becomes active at the time of blood pressure measurement (hereinafter also referred to as an “active measurement mode”) from the normal mode to a consecutive measurement mode (corresponding to (4) in FIG. 1). The consecutive measurement mode is a measurement mode in which a series of processes from a start to an end of blood pressure measurement performed in the normal mode are consecutively repeated a plurality of times (for example, three times). Thus, a blood pressure value obtained from blood pressure measurement in the consecutive measurement mode (for example, an average blood pressure value of three measurement operations) is a more reliable value than the blood pressure value obtained from one measurement operation in the normal mode. That is, it can be said that the consecutive measurement mode is a measurement mode in which the reliability of a measurement result (in this case, a blood pressure value) is higher than that in the normal mode (a more proper blood pressure value can be obtained). Note that, if no arrhythmia has occurred, the active measurement mode remains in the normal mode.

Subsequently, the blood-pressure gauge 100 performs blood pressure measurement using the consecutive measurement mode at the time of the next blood pressure measurement (corresponding to (5) in FIG. 1).

According to the above application example, if an arrhythmia has occurred at the time of blood pressure measurement in the normal mode (corresponding to (3) in FIG. 1), there is a possibility that the blood pressure value obtained in the blood pressure measurement is unreliable. Therefore, in (4) of FIG. 1, the active measurement mode is switched from the normal mode to the consecutive measurement mode that has high reliability. Then, as indicated by (5) in FIG. 1, the blood-pressure gauge 100 performs blood pressure measurement using the consecutive measurement mode.

As a result, a blood pressure of a user in whom an arrhythmia has been detected while sleeping will be measured by using the more reliable measurement mode at the time of the next blood pressure measurement. Therefore, it is possible to obtain a highly reliable measurement result even for a user in whom an arrhythmia has been detected.

On the other hand, for a user in whom an arrhythmia has not been detected while sleeping, since a highly reliable measurement result is obtained even in the normal mode, the blood pressure measurement is performed in the normal mode even in the next blood pressure measurement. In this case, the user can avoid a troublesome situation, such as an increase in the time required for measurement.

As described above, according to the blood-pressure gauge 100 of the present embodiment, it is possible to improve the reliability of measurement results of blood pressure measurement that automatically starts.

### Configuration Example

### Appearance

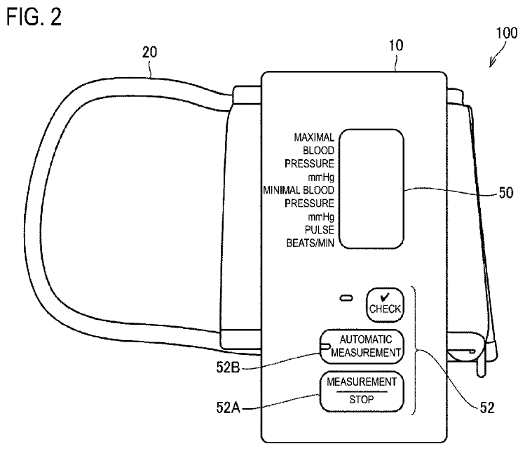

FIG. 2 is a diagram illustrating appearance of the blood-pressure gauge 100. The blood-pressure gauge 100 includes a cuff 20 for blood pressure measurement to be worn around a wrist as a measurement site, and a main body 10 integrally attached to the cuff 20.

The cuff 20 has an elongated band-like shape allowing it to wrap around a wrist in the circumferential direction. The cuff 20 contains a fluid bladder for compressing the wrist therein. Note that, in order to maintain the cuff 20 in an annular shape at all times, a curler having appropriate flexibility may be provided inside the cuff 20.

The main body 10 is integrally attached to a substantially central portion of the cuff 20 having a band shape in the longitudinal direction. The main body 10 has a flat, substantially rectangular parallelepiped shape along the outer circumferential surface of the cuff 20. The main body 10 is formed to be compact and thin not to interfere with sleep of the user. In addition, the corners of the main body 10 are rounded.

A display 50 serving as a display screen and an operation unit 52 for inputting an instruction from the user are provided on a surface (top surface) on the side farthest from the wrist on the outer surface of the main body 10.

The display 50 is constituted of, for example, a liquid crystal display (LCD) and displays various kinds of information in accordance with a control signal from a processor. In the example of FIG. 2, maximal blood pressure (unit: mmHg), minimal blood pressure (unit: mmHg), and pulse rate (unit: beats/minute) are displayed. Note that the display 50 may be an organic electro-luminescence (EL) display or may include light emitting diodes (LEDs).

The operation unit 52 inputs an operation signal corresponding to an instruction from a user to the processor. The operation unit 52 includes a measurement switch 52A for receiving a blood pressure measurement instruction (for example, a manual measurement instruction) from the user and an automatic measurement switch 52B for receiving an automatic measurement instruction.

Here, “manual measurement” means a measurement method in which, when a blood pressure measurement instruction is input by the measurement switch 52A, a blood pressure is measured according to the blood pressure measurement instruction. “Automatic measurement” means a measurement method in which blood pressure measurement is automatically started according to a predetermined schedule so that a blood pressure value can be measured while the user is sleeping. The “predetermined schedule” refers to, for example, a plan to measure a value at fixed times such as 1 AM, 2 AM, or 3 AM, a plan to measure a value once every two hours after the automatic measurement switch 52B is pressed, or the like. Therefore, the user may press the automatic measurement switch 52B if the user desires to automatically start blood pressure measurement even at night or during sleep.

When the measurement switch 52A is pressed, the measurement site is temporarily compressed by the cuff 20, and blood pressure measurement is performed in the oscillometric method. When the measurement switch 52A is pressed again during the blood pressure measurement (for example, during pressurization of the cuff 20), the blood pressure measurement is stopped.

In addition, when the automatic measurement switch 52B is pressed, blood pressure measurement in the oscillometric method is automatically started in accordance with a predetermined schedule. When the automatic measurement switch 52B is pressed again while the blood-pressure gauge 100 is performing automatic measurement, the blood-pressure gauge 100 stops the automatic measurement.

### Hardware Configuration

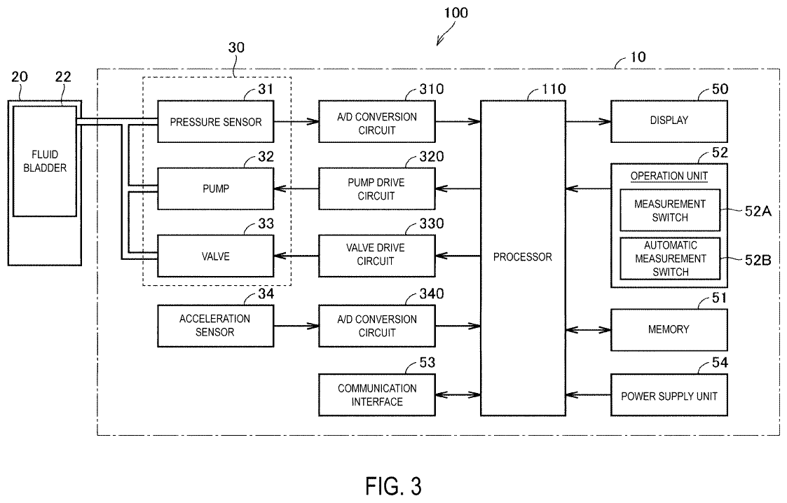

FIG. 3 is a block diagram illustrating a hardware configuration of the blood-pressure gauge 100. Referring to FIG. 3, the blood-pressure gauge 100 includes the main body 10 and the cuff 20 as main components. A fluid bladder 22 is contained in the cuff 20. The main body 10 includes a processor 110, an air system component 30 for blood pressure measurement, an acceleration sensor 34, A/D conversion circuits 310 and 340, a pump drive circuit 320, a valve drive circuit 330, the display 50, a memory 51, the operation unit 52, a communication interface 53, and a power supply unit 54.

The processor 110 is an arithmetic processing unit such as a central processing unit (CPU) or a multi processing unit (MPU). By reading and executing a program stored in the memory 51, the processor 110 realizes each of processing operations (steps) of the blood-pressure gauge 100 to be described below. For example, the processor 110 performs control to drive a pump 32 and a valve 33 according to an operation signal from the operation unit 52. In addition, the processor 110 calculates a blood pressure value using an algorithm for blood pressure calculation in the oscillometric method and displays the blood pressure value on the display 50.

The memory 51 is realized by a random access memory (RAM), a read-only memory (ROM), a flash memory, or the like. The memory 51 stores a program for controlling the blood-pressure gauge 100, data used to control the blood-pressure gauge 100, settings data for setting various functions of the blood-pressure gauge 100, data of measured blood pressure values, pulse rates, pulse wave intervals, and the like. In addition, the memory 51 is used as a working memory and the like for executing a program.

The air system component 30 supplies or discharges air to or from the fluid bladder 22 included in the cuff 20 through air piping. The air system component 30 includes a pressure sensor 31 for detecting pressure inside the fluid bladder 22, and the pump 32 and the valve 33 as an expansion/contraction mechanism unit for expanding and contracting the fluid bladder 22.

The pressure sensor 31 detects the pressure (cuff pressure) inside the fluid bladder 22 and outputs a signal (cuff pressure signal) corresponding to the detected pressure to the A/D conversion circuit 310. The pressure sensor 31 is, for example, a piezoresistive pressure sensor connected to the pump 32, the valve 33, and the fluid bladder 22 included in the cuff 20 via air piping. The pump 32 supplies air as a fluid to the fluid bladder 22 through the air piping in order to increase the cuff pressure. The valve 33 is opened and closed to control the cuff pressure by discharging air inside the fluid bladder 22 through the air piping or sealing the air in the fluid bladder 22.

The A/D conversion circuit 310 converts an output value of the pressure sensor 31 (for example, a voltage value corresponding to a change in electric resistance caused by a piezoresistive effect) from an analog signal to a digital signal and outputs the digital signal to the processor 110. The processor 110 acquires a signal representing the cuff pressure according to the output value of the A/D conversion circuit 310. The pump drive circuit 320 controls driving of the pump 32 based on a control signal from the processor 110. The valve drive circuit 330 controls opening and closing of the valve 33 based on a control signal from the processor 110.

The processor 110 performs blood pressure measurement by using a pressurization measurement method of measuring a blood pressure of the user based on a pulse wave signal in a pressurization process of increasing the cuff pressure or a depressurization measurement method of measuring a blood pressure of the user based on a pulse wave signal in a depressurization process of reducing the cuff pressure after the pressurization process of increasing the cuff pressure to a pressure higher than an estimated systolic blood pressure.

For example, at the time of measurement by the depressurization measurement method, the following operation is generally performed. The cuff is wrapped around the measurement site (wrist, arm, etc.) of the user, and during measurement, the pump 32 and the valve 33 are controlled such that the cuff pressure increases above the estimated systolic blood pressure, and then gradually decreases. In the depressurization process, the cuff pressure is detected by the pressure sensor 31, and the fluctuation of the arterial volume generated in the artery at the measurement site is extracted as a pulse wave signal. The maximal blood pressure (systolic blood pressure) and the minimal blood pressure (diastolic blood pressure) are calculated based on the change in amplitude of the pulse wave signal caused by the change in the cuff pressure at that time (mainly, rises and falls).

The A/D conversion circuit 340 converts the output of the acceleration sensor 34 from an analog signal to a digital signal and outputs the digital signal to the processor 110. For example, the acceleration sensor 34 is used to detect body motions of the user.

The display 50 displays various kinds of information including a blood pressure measurement result and the like based on a control signal from the processor 110. The communication interface 53 exchanges various kinds of information with an external device. The power supply unit 54 supplies power to the processor 110 and each piece of hardware.

The operation unit 52 inputs an operation signal corresponding to an instruction from the user to the processor 110. As described in FIG. 2, the operation unit 52 includes the measurement switch 52A and the automatic measurement switch 52B.

### Functional Configuration

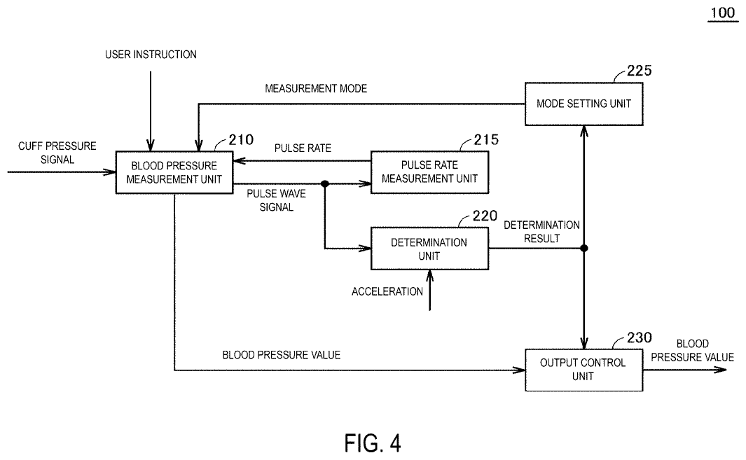

FIG. 4 is a block diagram illustrating a functional configuration of the blood-pressure gauge 100. Referring to FIG. 4, the blood-pressure gauge 100 includes, as main functional components, a blood pressure measurement unit 210, a pulse rate measurement unit 215, a determination unit 220, a mode setting unit 225, and an output control unit 230. Each of the functions is realized, for example, by the processor 110 of the blood-pressure gauge 1100 executing a program stored in the memory 51. Some or all of these functions may be configured to be realized by hardware.

The blood pressure measurement unit 210 controls the cuff pressure indicating the internal pressure of the cuff 20 according to a measurement start instruction (for example, a manual measurement instruction) from the user via the operation unit 52. Specifically, the blood pressure measurement unit 210 performs control to drive the pump 32 via the pump drive circuit 320 and to drive the valve 33 via the valve drive circuit 330. The valve 33 is opened and closed to control the cuff pressure, for example, by discharging air from the fluid bladder 222 or sealing air therein.

The blood pressure measurement unit 210 receives a cuff pressure signal detected by the pressure sensor 31 and extracts a pulse wave signal representing the pulse wave of the measurement site superimposed on the cuff pressure signal. That is, the blood pressure measurement unit 210 detects a pulse wave, which is a pressure component superimposed on the cuff pressure signal in synchronization with the user's heartbeat, from the cuff pressure signal.

The blood pressure measurement unit 210 calculates blood pressure information of the user based on the cuff pressure signal and the pulse wave signal superimposed on the cuff pressure signal. The blood pressure measurement unit 201 measures a blood pressure of a user using, for example, the oscillometric method.

On the other hand, the blood pressure measurement unit 210 automatically measures a blood pressure according to a predetermined schedule in response to pressing of the automatic measurement switch 52B. During automatic measurement, the blood pressure measurement unit 210 measures a blood pressure in one of a plurality of blood pressure measurement modes. The plurality of blood pressure measurement modes include the normal mode and a plurality of measurement modes M1 to M4 having higher reliability in measurement results than the normal mode. A measurement result includes a blood pressure value and a determination result of an arrhythmia.

The measurement mode M1 is a mode in which blood pressure measurement is consecutively repeated a plurality of times (for example, three times), and corresponds to the consecutive measurement mode described above.

The measurement mode M2 is a mode in which blood pressure measurement is performed by using the pressurization measurement method and in which the pressurization process in the pressurization measurement method is continued until the pulse rate equal to or higher than a threshold Th1 is acquired. When the measurement mode M2 is executed, the blood pressure measurement unit 210 measures a blood pressure of the user based on the pulse wave signal in a first pressurization process in which the cuff pressure is increased, and continues the first pressurization process until a pulse rate N1 of the user acquired based on a pulse wave signal in the first pressurization process reaches the threshold Th1 or higher. As a result, since a larger amount of pulse rates can be acquired at the time of blood pressure measurement in the measurement mode M2 than in the normal mode, a highly reliable arrhythmia determination result can be obtained.

The measurement mode M3 is a mode in which blood pressure measurement is performed by using the depressurization measurement method and in which the pressurization process in the depressurization measurement method is continued until the pulse rate equal to or higher than a threshold Th2 is acquired. When the measurement mode M3 is executed, the blood pressure measurement unit 210 measures a blood pressure of the user based on the pulse wave signal in the depressurization process of decreasing the cuff pressure after a second pressurization process to increase the cuff pressure to a pressure higher than the estimated systolic blood pressure, and continues the second pressurization process until a pulse rate N2 of the user acquired based on the pulse wave signal in the second pressurization process reaches the threshold Th2 or higher. As a result, since a larger amount of pulse rates can be acquired at the time of blood pressure measurement in the measurement mode M3 than in the normal mode, a highly reliable arrhythmia determination result can be obtained.

The measurement mode M4 is a mode in which a blood pressure is re-measured when the user makes a body motion during the blood pressure measurement. Detailed processing performed in the above five measurement modes (that is, the normal mode and the measurement modes M1 to M4) will be described below.

When the blood pressure measurement unit 210 performs blood pressure measurement using the pressurization measurement method, the pulse rate measurement unit 215 measures the pulse rate N1 of the user based on a pulse wave signal in the first pressurization process. In addition, when the blood pressure measurement is performed using the depressurization measurement method, the pulse rate measurement unit 215 measures the pulse rate N2 of the user based on a pulse wave signal in the second pressurization process.

The determination unit 220 determines whether any of a plurality of predetermined events has occurred in the user while the blood pressure measurement unit 210 was automatically measuring a blood pressure. The plurality of events include irregular pulse waves, arrhythmias, and body motions.

In an aspect, the determination unit 220 determines whether an irregular pulse wave has occurred in the user based on a pulse wave signal obtained when the blood pressure measurement unit 210 is performing blood pressure measurement. A known method is used as a method for determining an irregular pulse wave. For example, the determination unit 220 determines that an irregular pulse wave has occurred if there is a pulse wave deviating by ±25% or greater from the average value of the pulse wave intervals acquired while blood pressure measurement was performed.

In another aspect, the determination unit 220 determines whether an arrhythmia (a type of irregular pulse wave) has occurred in the user based on a pulse wave signal obtained when the blood pressure measurement unit 210 is performing blood pressure measurement. A known method is used as a method for determining an arrhythmia. For example, the determination unit 220 determines whether an arrhythmia has occurred based on the regularity of pulse wave intervals. Therefore, a sufficient number of pulse wave intervals are required in order to determine an arrhythmia with high accuracy.

In still another aspect, the determination unit 220 determines whether a body motion has been detected based on a pulse wave signal acquired during blood pressure measurement. For example, if the difference between the magnitude (amplitude) of a certain pulse wave and the magnitudes of pulse waves before and after the certain pulse wave is greater than or equal to a predetermined value, the determination unit 220 determines that a body motion has been made. Alternatively, the determination unit 220 determines that a body motion has been made when the cuff pressure has changed by a predetermined pressure or more within a predetermined period of time (for example, within one second).

Typically, when the blood pressure measurement unit 210 performs blood pressure measurement by using the pressurization measurement method, the determination unit 220 determines whether an irregular pulse wave, an arrhythmia, and a body motion have occurred based on a pulse wave signal in the pressurization process of the pressurization measurement method. When the blood pressure measurement unit 210 performs blood pressure measurement by using the depressurization measurement method, the determination unit 220 determines whether an irregular pulse wave, an arrhythmia, and a body motion have occurred based on a pulse wave signal in the depressurization process of the depressurization measurement method.

Note that the determination unit 220 may determine whether the user has made a body motion based on a change in the output value obtained from the acceleration sensor 34. The determination unit 220 obtains the average value of the outputs of the acceleration sensor 34 for each unit period (for example, one second or several seconds), and further obtains a variation amount by which the acceleration output at each time during the unit period has varied with respect to the average value. Then, the determination unit 220 determines that a body motion has been made when the square root of the sum of squares of the variation amounts exceeds a predetermined threshold.

The mode setting unit 225 sets an active measurement mode to be performed by the blood pressure measurement unit 210 from among the plurality of blood pressure measurement modes based on the determination result of the determination unit 220. Specifically, the mode setting unit 225 sets the normal mode as the active measurement mode when none of a plurality of events (for example, irregular pulse waves, arrhythmias, and body motions) has occurred. The mode setting unit 225 sets any one of the first to fourth measurement modes as the active measurement mode when any one of the events has occurred. Hereinafter, the first to fourth measurement modes are also referred to as measurement modes M1 to M4, respectively.

First, a configuration for setting a measurement mode based on whether an arrhythmia has occurred will be described. In this case, the mode setting unit 225 sets the normal mode or the measurement mode M1 as the active measurement mode based on whether an arrhythmia has occurred in the user.

To be more specific, the mode setting unit 225 sets the normal mode as the active measurement mode when no arrhythmia has occurred, and sets the measurement mode M1 as the active measurement mode when an arrhythmia has occurred. In another example, if an arrhythmia has occurred, the mode setting unit 225 may set the measurement mode M1 as the active measurement mode when the number of times an arrhythmia has occurred in the most recent specified number of times (for example, five times) of blood pressure measurement including the current blood pressure measurement is equal to or greater than a reference number of times (for example, three times).

In still another example, if an arrhythmia has occurred in the current blood pressure measurement, the mode setting unit 225 may set the measurement mode M1 as the active measurement mode when arrhythmias have occurred in the most recent predetermined number of times (for example, two times) of the blood pressure measurement including the current blood pressure measurement. In other words, the mode setting unit 225 may set the measurement mode M1 as the active measurement mode when arrhythmias have consecutively occurred a predetermined number of times.

Next, a configuration for setting a measurement mode based on whether an irregular pulse wave has occurred will be described. In this case, the mode setting unit 225 sets the normal mode or one of the measurement modes M1 to M3 as the active measurement mode based on whether an irregular pulse wave has occurred in the user.

To be specific, the mode setting unit 225 sets the normal mode as the active measurement mode when no irregular pulse waves have occurred, and sets one of the measurement modes M1 to M3 when an irregular pulse wave has occurred. For example, one of the measurement modes M1 to M3 is selected in accordance with patterns X1 to X3 set in the blood-pressure gauge 100. When the pattern X1 has been set, the mode setting unit 225 selects (sets) the measurement mode M1 when an irregular pulse wave has occurred. Similarly, the mode setting unit 225 selects the measurement mode M2 when the pattern X2 has been set, and selects the measurement mode M3 when the pattern X3 has been set.

When any one of the measurement modes M1 to M3 has been set by the mode setting unit 225 as described above, the blood pressure measurement unit 210 determines whether a predetermined period has elapsed since the previous blood pressure measurement. When the predetermined period has not elapsed, the blood pressure measurement unit 210 performs blood pressure measurement in accordance with the set measurement mode among the measurement modes M1 to M3. When the predetermined period has elapsed, the mode setting unit 225 switches the active measurement mode to the normal mode. Thus, the blood pressure measurement unit 210 performs blood pressure measurement in the normal mode.

Note that when the blood pressure measurement unit 210 performs blood pressure measurement in the measurement mode M2, the determination unit 220 further determines whether an arrhythmia has occurred in the user based on a pulse wave signal in the first pressurization process during the blood pressure measurement. In addition, when the blood pressure measurement unit 210 performs blood pressure measurement in the measurement mode M3, the determination unit 220 further determines whether an arrhythmia has occurred in the user based on a pulse wave signal in the depressurization process during the blood pressure measurement.

Next, a configuration for setting a measurement mode based on whether a body motion has been made will be described. In this case, the mode setting unit 225 sets the normal mode or the measurement mode M4 as the active measurement mode based on whether the user has made a body motion.

To be more specific, the mode setting unit 225 sets the normal mode as the active measurement mode when no body motion has been made in the current blood pressure measurement, and sets the measurement mode M4 as the active measurement mode when a body motion has been made in the current blood pressure measurement and the cumulative number of times of blood pressure measurement is less than J times (for example, three times). When the measurement mode M4 is set, the blood pressure measurement unit 210 re-measures the blood pressure. The cumulative number of measurement operations is a cumulative number of times of blood pressure measurement performed after the blood pressure measurement unit 210 starts automatic measurement.

The output control unit 230 displays the measurement result of the blood pressure measurement unit 210 (for example, systolic blood pressure and diastolic blood pressure values) and the determination result of the determination unit 220 (for example, the determination result of the occurrence of arrhythmias, irregular pulse waves, or body motions) on the display 50. Note that the output control unit 230 may transmit the measurement result and the determination result to an external device via the communication interface 53, or may output sound via a speaker (not illustrated).

### Processing Procedure

### Mode Setting Process According to Occurrence of Arrhythmia (1)

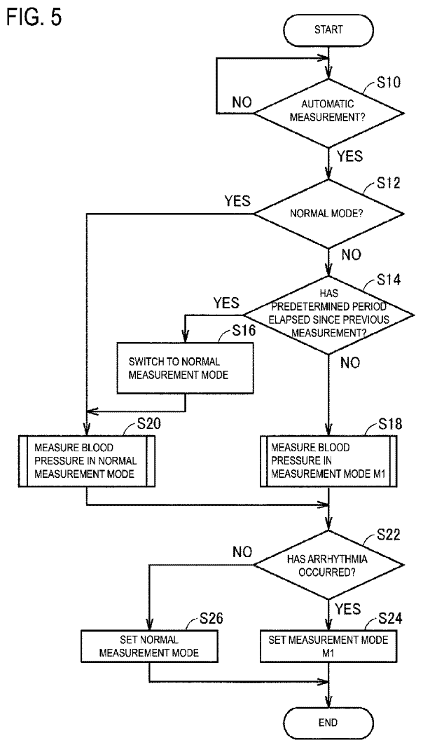

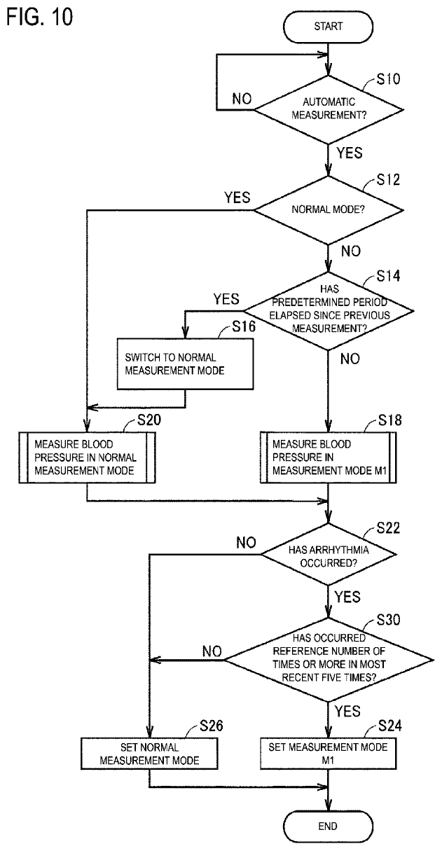

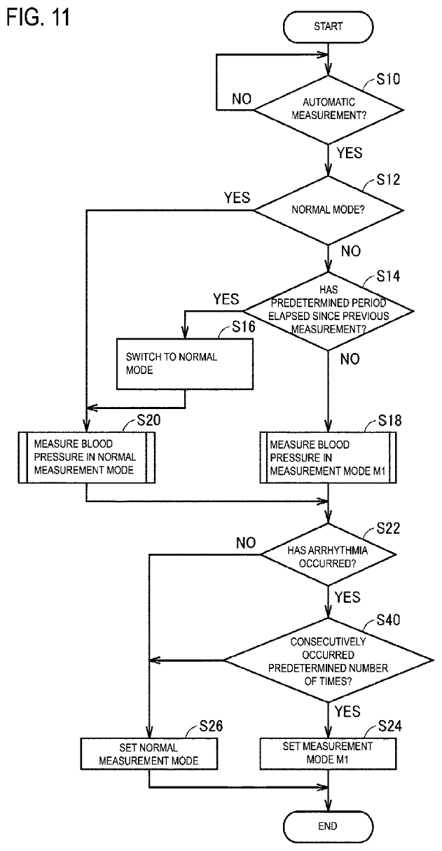

FIG. 5 is a flowchart showing an example of a setting process procedure for a measurement mode according to the occurrence of an arrhythmia. It is assumed that a user is wearing the cuff 20 of the blood-pressure gauge 100 at the start point of the process shown in FIG. 5. In FIG. 5, the normal mode or the measurement mode M1 is set as the active measurement mode. This also applies to FIGS. 10 and 11 to be described below.

Referring to FIG. 5, the processor 110 determines whether an automatic measurement start instruction has been received (i.e., whether selection of the automatic measurement switch 52B has been received) (step S10). If the start instruction has not been received (NO in step S10), the processor 110 repeats step S10. If the start instruction has been received (YES in step S10), the processor 110 determines whether the currently set active measurement mode is the normal mode (step S12).

If the active measurement mode is the normal mode (YES in step S12), the processor 110 measures a blood pressure in the normal mode (step S20). The blood pressure measurement process in the normal mode will be described below.

If the active measurement mode is not the normal mode (i.e., the active measurement mode is the measurement mode M1) (NO in step S12), the processor 110 determines whether a predetermined period (for example, a predetermined number of days such as one day or two days) has elapsed from the previous blood pressure measurement (step S14). If the predetermined period has elapsed (YES in step S14), the processor 110 switches the active measurement mode to the normal mode (step S16), and measures the blood pressure in the normal mode (step S20). If the predetermined period has not elapsed (NO in step S14), the processor 110 measures the blood pressure in the measurement mode M1 (step S18). The blood pressure measurement process in the measurement mode M1 will be described below.

Next, the processor 110 determines whether an arrhythmia has occurred based on a pulse wave signal obtained in the blood pressure measurement in step S18 or S20 (step S22). If an arrhythmia has occurred (YES in step S22), the processor 110 sets the measurement mode M1 as the active measurement mode (step S24). If no arrhythmia has occurred (NO in step S22), the processor 110 sets the normal mode as the active measurement mode (step S26).

Here, the blood pressure measurement process in the normal mode in step S20 of FIG. 5 will be described.

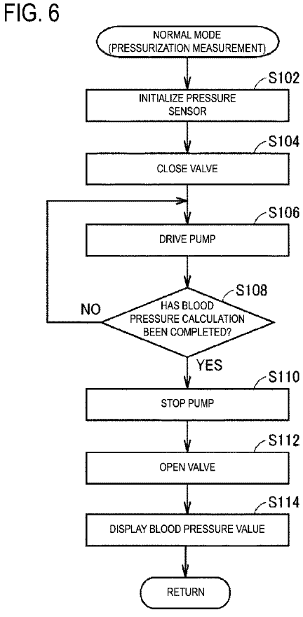

FIG. 6 is a flowchart showing an example of the blood pressure measurement process in the normal mode of the blood-pressure gauge 100. The process shown in FIG. 6 is a process of measuring a blood pressure by using the pressurization measurement method.

Referring to FIG. 6, the processor 110 of the blood-pressure gauge 100 initializes the pressure sensor 31 (step S102). To be more specific, the processor 110 initializes the processing memory area, turns off (stops) the pump 32, and adjusts the pressure sensor 31 to 0 mmHg (sets the atmospheric pressure to 0 mmHg) with the valve 33 being opened.

Next, the processor 110 closes the valve 33 via the valve drive circuit 330 (step S104), and turns on (activates) the pump 32 via the pump drive circuit 320 to start pressurization of the cuff 20 (fluid bladder 22) (step S106). At this time, the processor 110 controls the pressurization rate of the cuff pressure which is the pressure inside the fluid bladder 22 based on the output of the pressure sensor 31 while supplying air from the pump 32 to the fluid bladder 22 through the air piping.

Next, the processor 110 extracts a pulse wave signal from a cuff pressure signal detected by the pressure sensor 31, attempts to calculate the maximal blood pressure (systolic blood pressure) and the minimal blood pressure (diastolic blood pressure) based on the pulse wave signal, and determines whether the blood pressure calculation has been completed (step S108).

If the blood pressure calculation has not been completed yet due to a lack of data (NO in step S108), the processor 110 repeats the processing of steps S106 and S108 unless the cuff pressure reaches a predetermined upper pressure limit (e.g., 300 mmHg). If the blood pressure calculation has been completed (YES in step S108), the processor 110 performs control to stop the pump 32 (step S110), open the valve 33 (step S112), and discharge the air inside the cuff 20. The processor 110 displays the blood pressure value measured in the blood pressure measurement on the display 50 (step S114).

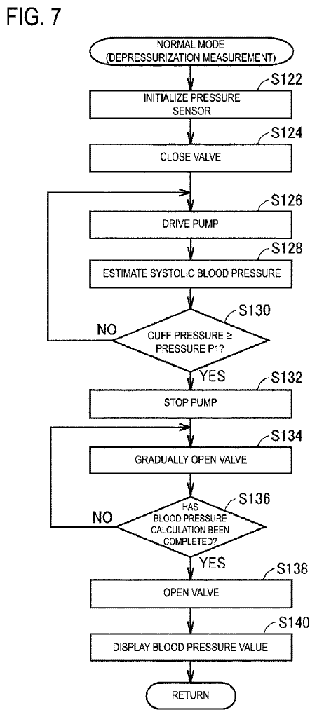

FIG. 7 is a flowchart showing another example of the blood pressure measurement process in the normal mode of the blood-pressure gauge 100. The process shown in FIG. 7 is a process of measuring a blood pressure by using the depressurization measurement method.

Referring to FIG. 7, since the processing of steps S122 to S126 is the same as the processing of steps S102 to S106 of FIG. 6, the detailed description thereof is not provided.

The processor 110 estimates the systolic blood pressure based on a pulse wave signal obtained during pressurization (step S128). The processor 110 determines whether the cuff pressure has reached a pressure P1 or greater (step S130). Typically, the pressure P1 is set to a value higher than the estimated systolic blood pressure value by a fixed value (e.g., 40 mmHg).

If the cuff pressure is lower than the pressure P1 (NO in step S130), the processor 110 returns to step S126. If the cuff pressure is equal to or higher than the pressure P1 (YES in step S130), the processor 110 stops the pump 32 (step S132) and performs control to gradually open the valve 33 (step S134). As a result, the cuff pressure gradually decreases from the pressurization process to the depressurization process.

In the depressurization process, the processor 110 extracts a pulse wave signal from the cuff pressure signal detected by the pressure sensor 31, attempts to calculate the systolic blood pressure and the diastolic blood pressure based on the pulse wave signal, and determines whether the blood pressure calculation has been completed (step S136). If the blood pressure calculation has not been completed (NO in step S136), the processor 110 repeats the processing of steps S134 and S136. If the blood pressure calculation has been completed (YES in step S136), the processor 110 performs control to fully open the valve 33 (step S138) and rapidly discharge the air inside the cuff 20. The processor 110 displays the blood pressure value measured in the blood pressure measurement on the display 50 (step S140).

Next, the blood pressure measurement process in the measurement mode M1 in step S18 of FIG. 5 will be described.

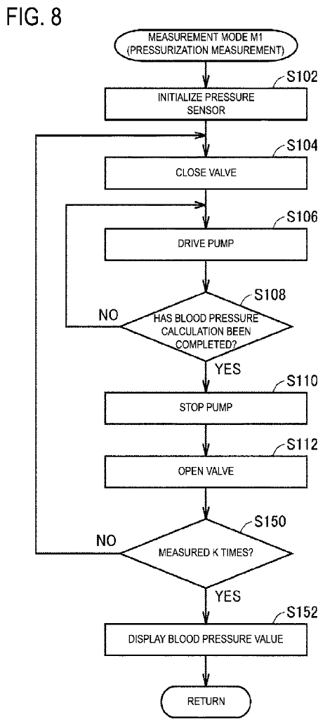

FIG. 8 is a flowchart showing an example of the blood pressure measurement process in the measurement mode M1 of the blood-pressure gauge 100. The process shown in FIG. 8 is a process of measuring a blood pressure by using the pressurization measurement method.

Referring to FIG. 8, since the processing of steps S102 to S112 is the same as the processing of FIG. 6, the detailed description thereof is not provided.

The processor 110 opens the valve 33 (step S112), and determines whether a series of blood pressure measurement processes from step S104 to step S112 have been consecutively performed K times (where, K≥2) (step S150). If the process has not been performed K times (NO in step S150), the processor 110 returns to the processing of step S104. If the process has been performed K times (YES in step S150), the processor 110 displays the measured blood pressure value on the display 50 (step S152). At this time, the processor 110 may display each blood pressure value of the K times or may display the average value of the blood pressure values of the K times.

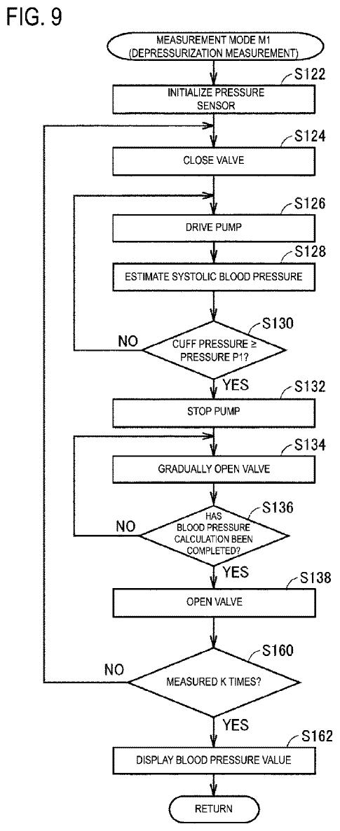

FIG. 9 is a flowchart showing another example of the blood pressure measurement process in the measurement mode M1 of the blood-pressure gauge 100. The process shown in FIG. 9 is a process of measuring a blood pressure by using the depressurization measurement method.

Referring to FIG. 9, since the processing of steps S122 to S138 is the same as the processing of FIG. 7, the detailed description thereof is not provided.

The processor 110 fully opens the valve 33 (step S138), and determines whether a series of blood pressure measurement processes from step S124 to step S138 have been consecutively performed K times (where, K≥2) (step S160). If the process has not been performed K times (NO in step S160), the processor 110 returns to the processing of step S124. If the process has been performed K times (YES in step S160), the processor 110 displays the measured blood pressure value on the display 50 (step S162). At this time, the processor 110 may display each blood pressure value of the K times or may display the average value of the blood pressure values of the K times.

According to the flowchart of FIG. 5, for a user to whom an arrhythmia has occurred during automatic measurement, a blood pressure is measured by using the measurement mode M1 for performing consecutive measurement a plurality of times in the next blood pressure measurement, and blood pressure values for a plurality of times or the average thereof can be obtained. Therefore, the blood-pressure gauge 100 can perform highly reliable blood pressure measurement even for such a user.

### Mode Setting Process According to Occurrence of Arrhythmia (2)

FIG. 10 is a flowchart showing another example of the setting process procedure for a measurement mode according to the occurrence of an arrhythmia.

Referring to FIG. 10, since the processing of steps S10 to S22 is the same as the processing of FIG. 5, the detailed description thereof is not provided.

If it is determined that no arrhythmia has occurred (NO in step S22), the processor 110 sets the normal mode as the active measurement mode (step S26). If it is determined that an arrhythmia has occurred (YES in step S22), the processor 110 determines whether the number of times an arrhythmia has occurred in the most recent specified number of times (for example, five times) of blood pressure measurement including the current blood pressure measurement is equal to or greater than a reference number of times (for example, three times) (step S30).

If the number of arrhythmias is equal to or greater than the reference number (YES in step S30), the processor 110 sets the measurement mode M1 as the active measurement mode (step S24). If the number of arrhythmias is less than the reference number (NO in step S30), the processor 110 sets the normal mode as the active measurement mode (step S26).

### Mode Setting Process According to Occurrence of Arrhythmia (3)

FIG. 11 is a flowchart showing another example of the setting process procedure for a measurement mode according to the occurrence of an arrhythmia.

Referring to FIG. 11, since the processing of steps S10 to S22 is the same as the processing of FIG. 5, the detailed description thereof is not provided.

If it is determined that no arrhythmia has occurred (NO in step S22), the processor 110 sets the normal mode as the active measurement mode (step S26). If it is determined that an arrhythmia has occurred (YES in step S22), the processor 110 determines whether arrhythmias have consecutively occurred a predetermined number of times (for example, two times) (step S40). If the predetermined number of times is two times, the processor 110 determines whether an arrhythmia has occurred even in the previous blood pressure measurement. If the predetermined number of times is three times, the processor 110 determines whether an arrhythmia has occurred even in the previous blood pressure measurement and the blood pressure measurement before that.

If arrhythmias have consecutively occurred the predetermined number of times (YES in step S40), the processor 110 sets the measurement mode M1 as the active measurement mode (step S24). If no arrhythmias have consecutively occurred the predetermined number of times (NO in step S40), the processor 110 sets the normal mode as the active measurement mode (step S26).

### Mode Setting Process According to Occurrence of Irregular Pulse Wave (1)

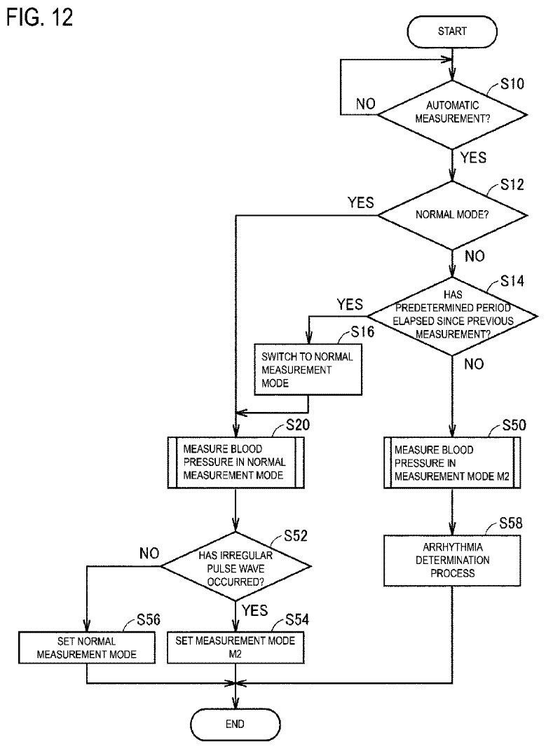

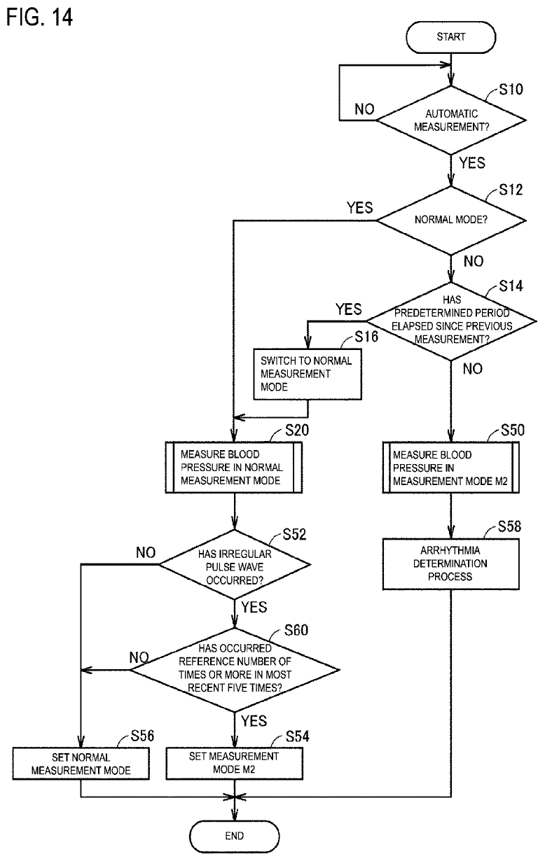

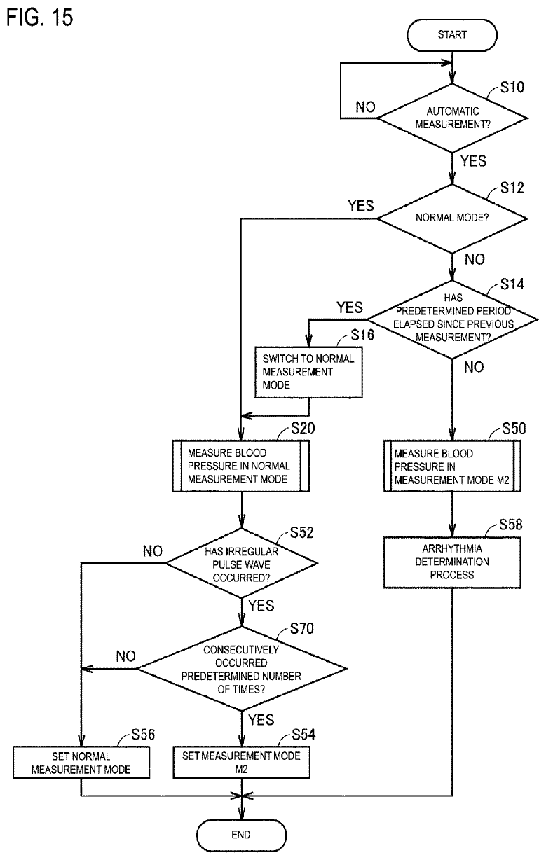

FIG. 12 is a flowchart showing an example of a setting process procedure for a measurement mode according to the occurrence of irregular pulse waves. In FIG. 12, the normal mode or the measurement mode M2 is set as an active measurement mode. This also applies to FIGS. 14 and 15 to be described below.

Referring to FIG. 12, since the processing of steps S10 to S16 and S20 is the same as the processing of FIG. 5, the detailed description thereof is not provided. Note that, if the currently set active measurement mode is not the normal mode in step S12 (NO in step S12), the processor 110 determines that the active measurement mode is the measurement mode M2.

If it is determined that the predetermined period has not elapsed from the previous blood pressure measurement (NO in step S14), the processor 110 measures the blood pressure in the measurement mode M2 (step S50). The blood pressure measurement process in the measurement mode M2 will be described below. Next, the processor 110 performs an arrhythmia determination process (step S58). To be more specific, the processor 110 determines whether an arrhythmia has occurred based on a pulse wave signal obtained in the pressurization process during the blood pressure measurement in the measurement mode M2. In this case, the processor 110 may display the arrhythmia determination result on the display 50.

In addition, the processor 110 determines whether an irregular pulse wave has occurred based on the pulse wave signal obtained in the blood pressure measurement in step S20 (step S52). If an irregular pulse wave has occurred (YES in step S52), the processor 110 sets the measurement mode M2 as the active measurement mode (step S54). If no irregular pulse wave has occurred (NO in step S52), the processor 110 sets the normal mode as the active measurement mode (step S56).

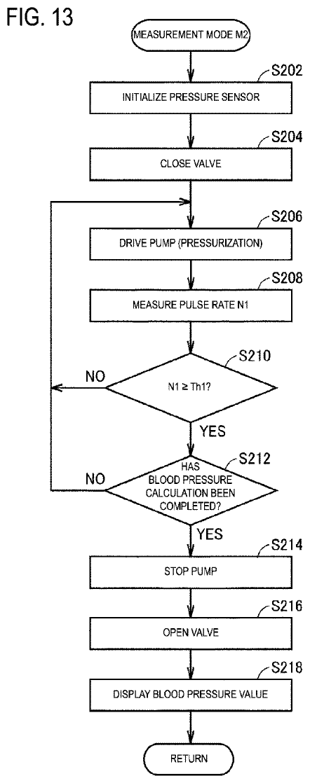

Here, the blood pressure measurement process in the measurement mode M2 in step S50 of FIG. 12 will be described. FIG. 13 is a flowchart showing an example of the blood pressure measurement process in the measurement mode M2 of the blood-pressure gauge 100. The process shown in FIG. 13 is a process of measuring a blood pressure by using the pressurization measurement method.

Referring to FIG. 13, since the processing of steps S202 to S206 is the same as the processing of steps S102 to S106 of FIG. 6, the detailed description thereof is not provided.

The processor 110 measures (counts) the pulse rate N1 based on a pulse wave signal extracted from a cuff pressure signal detected by the pressure sensor 31 in the pressurization process (step S208). The processor 110 determines whether the pulse rate N1 is equal to or higher than the threshold Th1 (step S210). If the pulse rate N1 is lower than the threshold Th1 (NO in step S210), the processor 110 returns to the processing of step S206 and continues to pressurize the cuff 20 as long as the cuff pressure does not reach the upper pressure limit Pmax (for example, 300 mmHg). If the pulse rate N1 is equal to or higher than the threshold Th1 (YES in step S210), the processor 110 attempts to calculate the maximal blood pressure (systolic blood pressure) and the minimal blood pressure (diastolic blood pressure), and determines whether the blood pressure calculation has been completed (step S212).

If the blood pressure calculation has not been completed (NO in step S212), the processor 110 repeats the processing of steps S206 to S212 to continue the pressurization process as long as the cuff pressure does not reach the predetermined upper pressure limit Pmax.

If the blood pressure calculation has been completed (YES in step S212), the processor 110 performs control to stop the pump 32 (i.e., stop the pressurization process) (step S214), open the valve 33 (step S216), and discharge the air inside the cuff 20. The processor 110 displays the blood pressure value obtained in step S212 on the display 50 (step S218).

The following processing may be performed instead of the processing of steps S208 to S212 described above.

In particular, after step S206, the processor 110 attempts to calculate the systolic blood pressure and diastolic blood pressure and determines whether the blood pressure calculation has been completed. If the blood pressure calculation has not been completed, the processor 110 repeats the processing from step S206 and continues the pressurization process as long as the cuff pressure does not reach the upper pressure limit Pmax.

If the blood pressure calculation has been completed, the processor 110 determines whether the cuff pressure is equal to or higher than the pressure P1 that is higher than the measured systolic blood pressure. Typically, the pressure P1 is set to a value higher than the systolic blood pressure by a predetermined value (e.g., 40 mmHg). If the cuff pressure is lower than the pressure P1, the processor 110 returns to step S206 and continues to pressurize the cuff 20. If the cuff pressure is equal to or higher than the pressure P1, the processor 110 measures the pulse rate N1 based on the pulse wave signal in the pressurization process.

The processor 110 determines whether the pulse rate N1 is equal to or higher than the threshold Th1. If the pulse rate N1 is lower than the threshold Th1, the processor 110 returns to the processing of step S206 and continues to pressurize the cuff 20 as long as the cuff pressure does not reach the upper pressure limit Pmax. If the pulse rate N1 is equal to or higher than the threshold Th1, the processor 110 performs the processing of step S214. The processing from step S214 is similar to that of in FIG. 13.

According to the flowchart of FIG. 12 described above, for a user to whom an irregular pulse wave has occurred during automatic measurement, blood pressure measurement is performed in the measurement mode M2 in which the pressurization process is continued until the pulse rate N1 becomes equal to or higher than the threshold Th1 in the next blood pressure measurement. Therefore, in the process of measuring the blood pressure, pulse rates sufficient for accurately detecting determination of an arrhythmia can be acquired, and a sufficient number of pulse wave intervals can be obtained. Therefore, the blood-pressure gauge 100 can more accurately determine an arrhythmia after blood pressure measurement in the measurement mode M2.

For example, if no arrhythmia is detected based on a pulse wave signal obtained during blood pressure measurement in the measurement mode M2, there is a low possibility of arrhythmias occurring to the user. On the other hand, if an arrhythmia has been detected based on a pulse wave signal obtained during blood pressure measurement in the measurement mode M2, there is a high possibility of an arrhythmia occurring to the user, and thus it is possible to call the user's attention by issuing a warning, or the like. In this way, the blood-pressure gauge 100 can determine arrhythmias with high reliability.

Mode Setting Process according to Occurrence of Irregular Pulse Wave (2)

FIG. 14 is a flowchart showing another example of the setting process procedure for a measurement mode according to the occurrence of irregular pulse waves.

Referring to FIG. 14, since the processing of steps S10 to S52 and S58 is the same as the processing of FIG. 12, the detailed description thereof is not provided.

If it is determined that no irregular pulse wave has occurred (NO in step S52), the processor 110 sets the normal mode as the active measurement mode (step S56). If it is determined that an irregular pulse wave has occurred (YES in step S52), the processor 110 determines whether the number of times an irregular pulse wave has occurred in the most recent specific number of times (for example, five times) is equal to or greater than a reference number of times (for example, three times) (step S60).

If the number of irregular pulse waves is equal to or greater than the reference number (YES in step S60), the processor 110 sets the measurement mode M2 as the active measurement mode (step S54). If the number of irregular pulse waves is less than the reference number (NO in step S60), the processor 110 sets the normal mode as the active measurement mode (step S56).

### Mode Setting Process According to Occurrence of Irregular Pulse Wave (3)

FIG. 15 is a flowchart showing another example of the setting process procedure for a measurement mode according to the occurrence of irregular pulse waves.

Referring to FIG. 15, since the processing of steps S10 to S52 and S58 is the same as the processing of FIG. 12, the detailed description thereof is not provided.

If it is determined that no irregular pulse wave has occurred (NO in step S52), the processor 110 sets the normal mode as the active measurement mode (step S56). If it is determined that an irregular pulse wave has occurred (YES in step S52), the processor 110 determines whether irregular pulse waves have consecutively occurred a predetermined number of times (for example, two times) (step S70).

If irregular pulse waves have consecutively occurred the predetermined number of times (YES in step S70), the processor 110 sets the measurement mode M2 as the active measurement mode (step S54). If no irregular pulse waves have consecutively occurred the predetermined number of times (NO in step S70), the processor 110 sets the normal mode as the active measurement mode (step S56).

### Another Example of Mode Setting According to Occurrence of Irregular Pulse Wave

Although the configuration in which the measurement mode M2 is set as the active measurement mode in the mode setting process of FIGS. 12, 14, and 15 when irregular pulse waves have occurred has been described, the configuration is not limited thereto. A configuration in which the measurement mode M1 or M3 is set as the active measurement mode when irregular pulse waves have occurred may be adopted. That is, a configuration in which the “measurement mode M2” in steps S12, S50, and S54 in FIGS. 12, 14, and 15 may be replaced with the “measurement mode M1” or the “measurement mode M3” may be adopted. The blood pressure measurement process in the measurement mode M1 is as described with reference to FIGS. 8 and 9. Here, a blood pressure measurement process in the measurement mode M3 will be described.

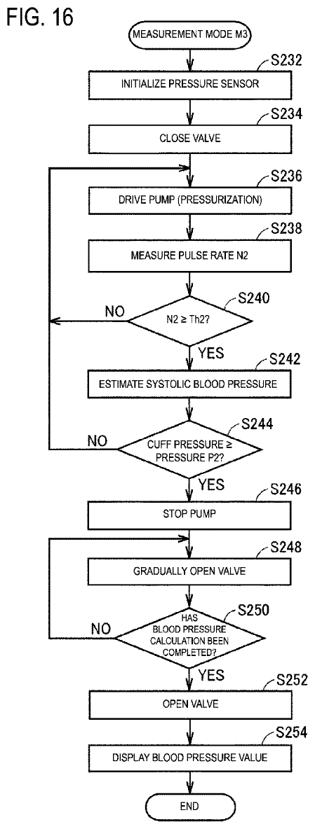

FIG. 16 is a flowchart showing an example of the blood pressure measurement process in the measurement mode M3 of the blood-pressure gauge 100. The process shown in FIG. 16 is a process of measuring a blood pressure by using the depressurization measurement method.

Referring to FIG. 16, since the processing of steps S232 to S236 is the same as the processing of steps S122 to S126 of FIG. 7, the detailed description thereof is not provided. Note that the pressurization process in the depressurization measurement mode is started by the processing of step S236. At this time, the blood-pressure gauge 100 controls the pressurization rate to be constant.

The processor 110 measures the pulse rate N2 based on a pulse wave signal obtained in the pressurization process in the depressurization measurement mode (step S238). The processor 110 determines whether the pulse rate N2 is equal to or higher than the threshold Th2 (step S240). If the pulse rate N2 is lower than the threshold Th2 (NO in step S240), the processor 110 returns to the processing of step S236 and continues to pressurize the cuff 20 as long as the cuff pressure does not reach the upper pressure limit Pmax. If the pulse rate N2 is equal to or higher than the threshold Th2 (YES in step S240), the processor 110 estimates the systolic blood pressure based on the pulse wave signal obtained in the pressurization process (step S242). The systolic blood pressure is estimated by using a known method. The processor 110 determines whether the cuff pressure is equal to or higher than a pressure P2 (step S244). Typically, the pressure P2 is set to a value higher than the estimated systolic blood pressure by a predetermined value (e.g., 40 mmHg).

If the cuff pressure is lower than the pressure P2 (NO in step S244), the processor 110 returns to step S236 and continues to pressurize the cuff 20. If the cuff pressure is equal to or higher than the pressure P2 (YES in step S244), the processor 110 stops the pump 32 (i.e., stops the pressurization process) (step S246) and performs control to gradually open the valve 33 (step S248). As a result, the cuff pressure gradually decreases from the pressurization process to the depressurization process. At this moment, the processor 110 controls the depressurization rate to be constant.

In the depressurization process, the processor 110 extracts a pulse wave signal from the cuff pressure signal detected by the pressure sensor 31, attempts to calculate the systolic blood pressure and the diastolic blood pressure based on the pulse wave signal, and determines whether the blood pressure calculation has been completed (step S250). If the blood pressure calculation has not been completed (NO in step S250), the processor 110 repeats the processing of steps S248 and S250. If the blood pressure calculation has been completed (YES in step S250), the processor 110 performs control to fully open the valve 33 (step S252) and rapidly discharge the air inside the cuff 20. The processor 110 displays the blood pressure value obtained in step S250 on the display 50 (step S254).

The following processing may be performed instead of the processing of steps S238 to S244 described above.

To be more specific, after step S236, the processor 110 estimates the systolic blood pressure based on the pulse wave signal obtained in the pressurization process in the depressurization measurement mode. Subsequently, the processor 110 determines whether the cuff pressure is equal to or higher than the pressure P2.

If the cuff pressure is lower than the pressure P2, the processor 110 returns to step S236 and continues to pressurize the cuff 20. If the cuff pressure is equal to or higher than the pressure P2, the processor 110 measures the pulse rate N2 based on the pulse wave signal obtained in the pressurization process. Next, the processor 110 determines whether the pulse rate N2 is equal to or higher than the threshold Th2.

If the pulse rate N2 is lower than the threshold Th2, the processor 110 returns to the processing of step S236 and continues to pressurize the cuff 20 as long as the cuff pressure does not reach the upper pressure limit Pmax. If the pulse rate N2 is equal to or higher than the threshold Th2, the processor 110 performs the processing of step S246. The processing from step S246 is similar to that of in FIG. 16.

If the “measurement mode M2” is replaced with the “measurement mode M3” in FIG. 12, for a user to whom an irregular pulse wave has occurred during automatic measurement, blood pressure measurement is performed in the measurement mode M3 in which the pressurization process is continued until the pulse rate N2 becomes equal to or higher than the threshold Th2 in the next blood pressure measurement. In the measurement mode M3, pressurization of the cuff is continued until the pulse rate N2 becomes equal to or higher than the threshold Th2 in the pressurization process, and thus the cuff pressure is set to be higher than usual when the depressurization process starts. As a result, assuming that the depressurization rate of the cuff is constant, the pulse rate obtained in the depressurization process becomes higher, and as a result, a sufficient number of pulse wave intervals can be obtained. Thus, as in the “measurement mode M2”, it is possible to more accurately determine arrhythmias after the blood pressure measurement in the “measurement mode M3”. That is, the blood-pressure gauge 100 can determine arrhythmias with high reliability.

When the “measurement mode M2” is replaced with the “measurement mode M1” in FIG. 12, blood pressure measurement is consecutively performed a plurality of times, and thus it is possible to acquire a larger amount of pulse rates in one measurement. Thus, the blood-pressure gauge 100 can determine arrhythmias with high reliability in this case as well.

### Mode Setting Process According to Occurrence of Body Motion

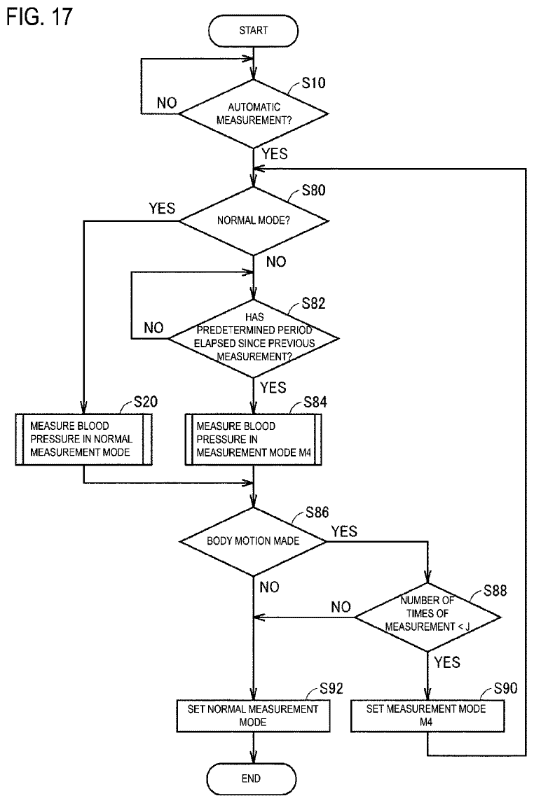

FIG. 17 is a flowchart showing an example of a setting process procedure for a measurement mode according to the occurrence of a body motion. In FIG. 17, the normal mode or the measurement mode M4 is set as the active measurement mode.

Referring to FIG. 17, since the processing of steps S10 and S20 is the same as the processing of FIG. 5, the detailed description thereof is not provided.

If an instruction to start automatic measurement has been received (YES in step S10), the processor 110 determines whether the currently set active measurement mode is the normal mode (step S80).

If the active measurement mode is the normal mode (YES in step S80), a blood pressure is measured in the normal mode (step S20). If the active measurement mode is not the normal mode (i.e., the active measurement mode is the measurement mode M4) (NO in step S80), the processor 110 determines whether a predetermined period (for example, a few dozens minutes) has elapsed from the previous blood pressure measurement (step S82). If the predetermined period has elapsed (NO in step S82), the processor 110 repeats the processing of step S82. If the predetermined period has elapsed (YES in step S82), the processor 110 measures the blood pressure in the measurement mode M4 (step S84). That is, the processor 110 re-measures the blood pressure according to the same blood pressure measurement process as that in the normal mode (for example, the flowchart of FIG. 6 or 7).

Subsequently, the processor 110 determines whether a body motion has been made by the user during the blood pressure measurement (step S86). If no body motion has been made (NO in step S86), the processor 110 sets the normal mode as the active measurement mode (step S92). If a body motion has been made (YES in step S86), the processor 110 determines whether the cumulative number of times of measurement from the start of automatic measurement is less than J times (for example, three times) (step S88).

If the cumulative number of times of measurement is equal to or greater than J times (NO in step S88), the processor 110 sets the normal mode as the active measurement mode (step S92). If the cumulative number of times of measurement is less than J times (YES in step S88), the processor 110 sets the measurement mode M4 as the active measurement mode (step S90), and returns to the processing of step S80. That is, the blood pressure is re-measured.

According to the flowchart of FIG. 17, the measurement mode M4 is set for a user who made a body motion during automatic measurement, and the blood pressure is automatically re-measured. Thus, the blood-pressure gauge 100 can measure the blood pressure with high reliability.

### OTHER EMBODIMENTS

(1) Although the configuration in which the measurement mode M1 is set in step S24 and the blood pressure is measured in the measurement mode M1 in step S18 in the next automatic measurement in FIGS. 5, 10, and 11 has been described in the above-described embodiment, the present invention is not limited thereto. For example, when the measurement mode M1 is set in step S24 in FIGS. 5, 10, and 11, the blood-pressure gauge 100 may measure the blood pressure in the measurement mode M1 immediately after step S24.

Likewise, when the measurement mode M2 is set in step S54 in FIGS. 12, 14, and 15, the blood-pressure gauge 100 may measure the blood pressure in the measurement mode M2 immediately after step S54. Note that, as described above, the measurement mode M1 or the measurement mode M3 may be set instead of the measurement mode M2.

According to the above-described configuration, when any event (for example, an arrhythmia, an irregular pulse wave, or a body motion) occurs to a user during automatic measurement, it is possible to immediately measure the blood pressure in the measurement mode with higher reliability with no need to wait for the next measurement chance.

(2) In the embodiments described above, a program that causes a computer to function and execute control such as that described in the flowcharts described above may be provided. Such a program can also be provided as a program product stored on a transient computer-readable recording medium attached to a computer, such as a flexible disk, a compact disc read only memory (CD-ROM), a secondary storage device, a main storage device, and a memory card. Alternatively, a program may be provided, which is stored on a recording medium such as a hard disk built into a computer. In addition, the program may also be provided by download via a network.

(3) The configuration given as an example of the embodiment described above is an example configuration of the present invention. The configuration can be combined with other known technology, and parts thereof may be omitted or modified within the scope of the present invention. Furthermore, the processes and configurations of other embodiments may be employed as appropriate to the embodiments described above.

### Supplementary Notes

As described above, the present embodiments include the following disclosures.

### Configuration 1

A blood-pressure gauge (100) for measuring a blood pressure by compressing a measurement site of a user with a cuff (20) includes a blood pressure measurement unit (210) that automatically performs blood pressure measurement in one of a plurality of blood pressure measurement modes according to a predetermined schedule, the plurality of blood pressure measurement modes including a normal measurement mode and a plurality of measurement modes having higher reliability in a measurement result than the normal measurement mode, and further includes a determination unit (220) that determines whether any of a plurality of predetermined events has occurred in the user while the blood pressure measurement unit (210) was measuring a blood pressure, and a mode setting unit (225) that sets an active measurement mode to be performed by the blood pressure measurement unit (210) from among the plurality of blood pressure measurement modes based on a determination result of the determination unit (220), in which the mode setting unit (225) sets the normal measurement mode as the active measurement mode when none of the plurality of events has occurred, and sets one of the plurality of measurement modes as the active measurement mode when one of the plurality of events has occurred.

### Configuration 2

The blood-pressure gauge (100) described in configuration 1, in which the plurality of measurement modes include a first measurement mode in which blood pressure measurement is consecutively performed a plurality of times or more, the determination unit (220) determines whether an arrhythmia has occurred in the user based on a pulse wave signal obtained when the blood pressure measurement unit (210) performs blood pressure measurement, and the mode setting unit (225) sets the normal measurement mode or the first measurement mode as the active measurement mode based on whether an arrhythmia has occurred.

### Configuration 3

The blood-pressure gauge (100) described in configuration 2, in which the mode setting unit (225) sets the normal measurement mode as the active measurement mode when no arrhythmia has occurred, and sets the first measurement mode as the active measurement mode when an arrhythmia has occurred.

### Configuration 4

The blood-pressure gauge (100) described in configuration 2, in which the mode setting unit (225) sets the normal measurement mode as the active measurement mode if no arrhythmia has occurred, and sets the first measurement mode as the active measurement mode when the number of times an arrhythmia has occurred in the most recent specified number of times of blood pressure measurement is equal to or greater than a reference number of times if an arrhythmia has occurred.

### Configuration 5

The blood-pressure gauge (100) described in configuration 2, in which the mode setting unit (225) sets the normal measurement mode as the active measurement mode when no arrhythmia has occurred, and sets the first measurement mode as the active measurement mode when arrhythmias have consecutively occurred a predetermined number of times.

### Configuration 6

The blood-pressure gauge (100) described in any one of configurations 2 to 5, in which, when the first measurement mode is set, the blood pressure measurement unit (210) determines whether a predetermined period has elapsed from previous blood pressure measurement, performs blood pressure measurement in the first measurement mode when the predetermined period has not elapsed, and performs blood pressure measurement in the normal measurement mode when the predetermined period has elapsed.

### Configuration 7

The blood-pressure gauge (100) described in any one of configurations 1 to 6, in which the plurality of measurement modes include a second measurement mode, the blood pressure measurement unit (210) measures a blood pressure of the user based on a pulse wave signal in a first pressurization process of increasing a cuff pressure indicating a pressure inside the cuff worn around a measurement site of the user in the normal measurement mode, the determination unit (220) determines whether an irregular pulse wave has occurred in the user based on the pulse wave signal in the first pressurization process, and the mode setting unit (225) sets the normal measurement mode as the active measurement mode when no irregular pulse wave has occurred, and sets the second measurement mode as the active measurement mode when an irregular pulse wave has occurred.

### Configuration 8

The blood-pressure gauge (100) described in configuration 7, in which the blood pressure measurement unit (210) measures a blood pressure of the user based on the pulse wave signal in the first pressurization process in the second measurement mode, and continues the first pressurization process until a first pulse rate of the user acquired based on the pulse wave signal in the first pressurization process reaches a threshold or higher, and the determination unit (220) determines whether an arrhythmia has occurred in the user based on the pulse wave signal in the first pressurization process.

### Configuration 9

The blood-pressure gauge (100) described in any one of configurations 1 to 8, in which the plurality of measurement modes include a third measurement mode, the blood pressure measurement unit (210) measures a blood pressure of the user based on a pulse wave signal in a depressurization process of reducing the cuff pressure after a second pressurization process of increasing a cuff pressure indicating a pressure inside the cuff worn around a measurement site of the user to a pressure higher than an estimated systolic blood pressure in the normal measurement mode, the determination unit (220) determines whether an irregular pulse wave has occurred in the user based on the pulse wave signal in the depressurization process, and the mode setting unit (225) sets the normal measurement mode as the active measurement mode when no irregular pulse wave has occurred, and sets the third measurement mode when an irregular pulse wave has occurred.

### Configuration 10

The blood-pressure gauge (100) described in configuration 9, in which the blood pressure measurement unit (210) measures a blood pressure of the user based on the pulse wave signal in the depressurization process in the third measurement mode after the second pressurization process, and continues the second pressurization process until a second pulse rate of the user acquired based on the pulse wave signal in the second pressurization process reaches a threshold or higher, and the determination unit (220) determines whether an arrhythmia has occurred in the user based on the pulse wave signal in the depressurization process.

### Configuration 11

The blood-pressure gauge (100) described in any one of configurations 1 to 10, in which the plurality of measurement modes include a fourth measurement mode in which a blood pressure is re-measured when the user makes a body motion, the determination unit (220) determines whether a body motion has been made based on a pulse wave signal obtained when the blood pressure measurement unit (210) performs blood pressure measurement, and the mode setting unit (225) sets the normal measurement mode or the fourth measurement mode as the active measurement mode based on whether a body motion has been made.

### Configuration 12

The blood-pressure gauge (100) described in configuration 11, in which the mode setting unit (225) sets the normal measurement mode as the active measurement mode when no body motion has been made in the current blood pressure measurement, and sets the fourth measurement mode as the active measurement mode when a body motion has been made in the current blood pressure measurement and the cumulative number of times of blood pressure measurement is less than a first number of times.

### Configuration 13

A method for measuring a blood pressure for a blood-pressure gauge (100) for measuring a blood pressure by compressing a measurement site of a user with a cuff (20) includes a step of automatically performing blood pressure measurement in one of a plurality of blood pressure measurement modes according to a predetermined schedule, the plurality of blood pressure measurement modes including a normal measurement mode and a plurality of measurement modes having higher reliability in a measurement result than the normal measurement mode, and further includes a step of determining whether any of a plurality of predetermined events has occurred in the user while the blood pressure was being measured, and a step of setting an active measurement mode to be performed in the performing step from among the plurality of blood pressure measurement modes based on a determination result of the determining step, in which, the setting step includes setting the normal measurement mode as the active measurement mode when none of the plurality of events has occurred, and setting one of the plurality of measurement modes as the active measurement mode when one of the plurality of events has occurred.

The embodiments described herein are illustrative in all respects and are not intended as limitations. The scope of the present invention is indicated not by the descriptions above but by the claims and includes all meaning equivalent to the scope and changes within the scope.

### REFERENCE NUMERALS LIST

10 Main body, 20 Cuff, 22 Fluid bladder, 30 Air system component, 31 Pressure sensor, 32 Pump, 33 Valve, 34 Acceleration sensor, 50 Display, 51 Memory, 52 Operation unit, 52A Measurement switch, 52B Automatic measurement switch, 53 Communication interface, 54 Power supply unit, 100 Blood-pressure gauge, 110 Processor, 210 Blood pressure measurement unit, 215 Pulse rate measurement unit, 220 Determination unit, 225 Mode setting unit, 230 Output control unit, 310, 340 A/D conversion circuit, 320 Pump drive circuit, 330 Valve drive circuit.

1. A blood-pressure gauge for measuring a blood pressure by compressing a measurement site of a user with a cuff, the blood-pressure gauge comprising:
a blood pressure measurement unit configured to automatically perform blood pressure measurement in one of a plurality of blood pressure measurement modes according to a predetermined schedule,
the plurality of blood pressure measurement modes including a normal measurement mode and a plurality of measurement modes having higher reliability in a measurement result than the normal measurement mode;
a determination unit configured to determine whether any of a plurality of predetermined events has occurred in the user while the blood pressure measurement unit was measuring a blood pressure; and
a mode setting unit configured to set an active measurement mode to be performed by the blood pressure measurement unit from among the plurality of blood pressure measurement modes based on a determination result of the determination unit,
wherein the mode setting unit sets the normal measurement mode as the active measurement mode when none of the plurality of events has occurred, and sets one of the plurality of measurement modes as the active measurement mode when one of the plurality of events has occurred,
the plurality of measurement modes include first to third measurement modes, and
the blood pressure measurement unit determines whether a predetermined period has elapsed from previous blood pressure measurement when one of the first to third measurement modes has been set as the active measurement mode,
performs blood pressure measurement in a measurement mode set as the active measurement mode among the first to third measurement modes when the predetermined period has not elapsed, and
sets the normal measurement mode as the active measurement mode and performs blood pressure measurement in the normal measurement mode when the predetermined period has elapsed,
the blood pressure measurement unit measures a blood pressure of the user based on a pulse wave signal in a first pressurization process of increasing a cuff pressure indicating a pressure inside the cuff worn around a measurement site of the user in the normal measurement mode,
the determination unit determines whether an irregular pulse wave has occurred in the user based on the pulse wave signal in the first pressurization process,
the mode setting unit sets the normal measurement mode as the active measurement mode when no irregular pulse wave has occurred, and
sets the second measurement mode as the active measurement mode when the number of times an irregular pulse wave has occurred in the most recent specified number of times of blood pressure measurement is equal to or greater than a reference number of times or when irregular pulse waves have consecutively occurred a predetermined number of times,
the blood pressure measurement unit measures a blood pressure of the user based on the pulse wave signal in the first pressurization process in the second measurement mode, and
continues the first pressurization process until a first pulse rate of the user acquired based on the pulse wave signal in the first pressurization process reaches a threshold or higher, and
the determination unit determines whether an arrhythmia has occurred in the user based on the pulse wave signal in the first pressurization process.

2. The blood-pressure gauge according to claim 1,
wherein the first measurement modes is a first measurement mode in which blood pressure measurement is consecutively performed a plurality of times or more,
the determination unit determines whether an arrhythmia has occurred in the user based on a pulse wave signal obtained when the blood pressure measurement unit performs blood pressure measurement, and
the mode setting unit sets the normal measurement mode or the first measurement mode as the active measurement mode based on whether an arrhythmia has occurred.

3. The blood-pressure gauge according to claim 2, wherein the mode setting unit sets the normal measurement mode as the active measurement mode when no arrhythmia has occurred, and sets the first measurement mode as the active measurement mode when an arrhythmia has occurred.

4. The blood-pressure gauge according to claim 2,
wherein the mode setting unit sets the normal measurement mode as the active measurement mode if no arrhythmia has occurred, and sets the first measurement mode as the active measurement mode when the number of times an arrhythmia has occurred in a most recent specified number of times of blood pressure measurement is equal to or greater than a reference number of times if an arrhythmia has occurred.

5. The blood-pressure gauge according to claim 2, wherein the mode setting unit sets the normal measurement mode as the active measurement mode when no arrhythmia has occurred, and sets the first measurement mode as the active measurement mode when arrhythmias have consecutively occurred a predetermined number of times.

6. A blood-pressure gauge for measuring a blood pressure by compressing a measurement site of a user with a cuff, the blood-pressure gauge comprising:
a blood pressure measurement unit configured to automatically perform blood pressure measurement in one of a plurality of blood pressure measurement modes according to a predetermined schedule,
the plurality of blood pressure measurement modes including a normal measurement mode and a plurality of measurement modes having higher reliability in a measurement result than the normal measurement mode;
a determination unit configured to determine whether any of a plurality of predetermined events has occurred in the user while the blood pressure measurement unit was measuring a blood pressure; and
a mode setting unit configured to set an active measurement mode to be performed by the blood pressure measurement unit from among the plurality of blood pressure measurement modes based on a determination result of the determination unit,
wherein the mode setting unit sets the normal measurement mode as the active measurement mode when none of the plurality of events has occurred, and sets one of the plurality of measurement modes as the active measurement mode when one of the plurality of events has occurred,
the plurality of measurement modes include first to third measurement modes,
the blood pressure measurement unit determines whether a predetermined period has elapsed from previous blood pressure measurement when one of the first to third measurement modes has been set as the active measurement mode,
performs blood pressure measurement in a measurement mode set as the active measurement mode among the first to third measurement modes when the predetermined period has not elapsed, and
sets the normal measurement mode as the active measurement mode and performs blood pressure measurement in the normal measurement mode when the predetermined period has elapsed,
the blood pressure measurement unit measures a blood pressure of the user based on a pulse wave signal in a depressurization process of reducing the cuff pressure after a second pressurization process of increasing a cuff pressure indicating a pressure inside the cuff worn around a measurement site of the user to a pressure higher than an estimated systolic blood pressure in the normal measurement mode,
the determination unit determines whether an irregular pulse wave has occurred in the user based on the pulse wave signal in the depressurization process, and
the mode setting unit sets the normal measurement mode as the active measurement mode when no irregular pulse wave has occurred, and sets the third measurement mode as the active measurement mode when the number of times an irregular pulse wave has occurred in the most recent specified number of times of blood pressure measurement is equal to or greater than a reference number of times or when irregular pulse waves have consecutively occurred a predetermined number of times,
the blood pressure measurement unit measures a blood pressure of the user based on the pulse wave signal in the depressurization process in the third measurement mode after the second pressurization process, and continues the second pressurization process until a second pulse rate of the user acquired based on the pulse wave signal in the second pressurization process reaches a threshold or higher, and
the determination unit determines whether an arrhythmia has occurred in the user based on the pulse wave signal in the depressurization process.

7. The blood-pressure gauge according to claim 1,
wherein the plurality of measurement modes include a fourth measurement mode in which a blood pressure is re-measured when the user makes a body motion,
the determination unit determines whether a body motion has been made based on a pulse wave signal obtained when the blood pressure measurement unit performs blood pressure measurement, and
the mode setting unit sets the normal measurement mode or the fourth measurement mode as the active measurement mode based on whether a body motion has been made.

8. The blood-pressure gauge according to claim 7,
wherein the mode setting unit sets the normal measurement mode as the active measurement mode when no body motion has been made in the current blood pressure measurement, and sets the fourth measurement mode as the active measurement mode when a body motion has been made in the current blood pressure measurement and a cumulative number of times of blood pressure measurement is less than a first number of times.

9. A method for measuring a blood pressure for a blood-pressure gauge for measuring a blood pressure by compressing a measurement site of a user with a cuff, the method for measuring a blood pressure comprising:
automatically performing blood pressure measurement in one of a plurality of blood pressure measurement modes according to a predetermined schedule,
the plurality of blood pressure measurement modes including a normal measurement mode and a plurality of measurement modes having higher reliability in a measurement result than the normal measurement mode;
determining whether any of a plurality of predetermined events has occurred in the user while the blood pressure was being measured; and
setting an active measurement mode to be performed in the performing from among the plurality of blood pressure measurement modes based on a determination result of the determining,
wherein, in the setting, the normal measurement mode is set as the active measurement mode when none of the plurality of events has occurred, and one of the plurality of measurement modes is set as the active measurement mode when one of the plurality of events has occurred,
the plurality of measurement modes include first to third measurement modes,
the performing includes determining whether a predetermined period has elapsed from previous blood pressure measurement when one of the first to third measurement modes has been set as the active measurement mode,
performing blood pressure measurement in a measurement mode set as the active measurement mode among the first to third measurement modes when the predetermined period has not elapsed, and
setting the normal measurement mode as the active measurement mode and performing blood pressure measurement in the normal measurement mode when the predetermined period has elapsed,
the performing includes measuring a blood pressure of the user based on a pulse wave signal in a first pressurization process of increasing a cuff pressure indicating a pressure inside the cuff worn around a measurement site of the user in the normal measurement mode,
the determining includes determining whether an irregular pulse wave has occurred in the user based on the pulse wave signal in the first pressurization process,
the setting includes setting the normal measurement mode as the active measurement mode when no irregular pulse wave has occurred, and
setting the second measurement mode as the active measurement mode when the number of times an irregular pulse wave has occurred in the most recent specified number of times of blood pressure measurement is equal to or greater than a reference number of times or when irregular pulse waves have consecutively occurred a predetermined number of times,
the performing includes measuring a blood pressure of the user based on the pulse wave signal in the first pressurization process in the second measurement mode, and
continuing the first pressurization process until a first pulse rate of the user acquired based on the pulse wave signal in the first pressurization process reaches a threshold or higher, and
the determining includes determining whether an arrhythmia has occurred in the user based on the pulse wave signal in the first pressurization process.

10. A method for measuring a blood pressure for a blood-pressure gauge for measuring a blood pressure by compressing a measurement site of a user with a cuff, the method for measuring a blood pressure comprising:
automatically performing blood pressure measurement in one of a plurality of blood pressure measurement modes according to a predetermined schedule,
the plurality of blood pressure measurement modes including a normal measurement mode and a plurality of measurement modes having higher reliability in a measurement result than the normal measurement mode;
determining whether any of a plurality of predetermined events has occurred in the user while the blood pressure was being measured; and
setting an active measurement mode to be performed in the performing from among the plurality of blood pressure measurement modes based on a determination result of the determining,
wherein the setting includes setting the normal measurement mode as the active measurement mode when none of the plurality of events has occurred, and setting one of the plurality of measurement modes as the active measurement mode when one of the plurality of events has occurred,
the plurality of measurement modes include first to third measurement modes,
the performing includes determining whether a predetermined period has elapsed from previous blood pressure measurement when one of the first to third measurement modes has been set as the active measurement mode,
performing blood pressure measurement in a measurement mode set as the active measurement mode among the first to third measurement modes when the predetermined period has not elapsed, and
setting the normal measurement mode as the active measurement mode and performing blood pressure measurement in the normal measurement mode when the predetermined period has elapsed,
the performing includes measuring a blood pressure of the user based on a pulse wave signal in a depressurization process of reducing a cuff pressure indicating a pressure inside the cuff worn around the measurement site of the user after a second pressurization process of increasing a cuff pressure to a pressure higher than an estimated systolic blood pressure in the normal measurement mode,
the determining includes determining whether an irregular pulse wave has occurred in the user based on the pulse wave signal in the depressurization process,
the setting includes setting the normal measurement mode as the active measurement mode when no irregular pulse wave has occurred, and
setting the third measurement mode as the active measurement mode when the number of times an irregular pulse wave has occurred in the most recent specified number of times of blood pressure measurement is equal to or greater than a reference number of times or when irregular pulse waves have consecutively occurred a predetermined number of times,
the performing includes measuring a blood pressure of the user based on the pulse wave signal in the depressurization process after the second pressurization process in the third measurement mode, and
continuing the second pressurization process until a second pulse rate of the user acquired based on a pulse wave signal in the second pressurization process reaches a threshold or higher, and
the determining includes determining whether an arrhythmia has occurred in the user based on the pulse wave signal in the depressurization process.
